(19) United States
(12) Patent Application Publication
(10) Pub. No.: US 2024/0102979 A1
(43) Pub. Date: Mar. 28, 2024

(54) Calibration concentration selection method for gas sensor array

(71) Applicant: University of Electronic Science and Technology of China, Chengdu (CN)

(72) Inventors: Huiling Tai, Chengdu (CN); Yadong Jiang, Chengdu (CN); Yilun Ma, Chengdu (CN); Zaihua Duan, Chengdu (CN); Yuanming Wu, Chengdu (CN); Zhen Yuan, Chengdu (CN)

(21) Appl. No.: 18/533,743

(22) Filed: Dec. 8, 2023

(30) Foreign Application Priority Data
Mar. 1, 2023 (CN) 202310183544.5

(51) Int. Cl. G01N 33/00

(52) U.S. Cl. CPC G01N 33/0006

(57) ABSTRACT

A calibration concentration selection method includes steps of: using a gas sensor array to obtain a concentration variation sequence and a response variation sequence of the gas mixture; constructing and training an AE-BP model; constructing VAE and identically distributing the response variation sequence; inputting the identically distributed response variation sequence into the trained AE-BP model to output a predicted concentration variation sequence; and then normalizing the predicted concentration variation sequence to generate a target concentration variation sequence; sorting a target concentration variation sequence and calculating a response gradient sequence; processing the response gradient sequence for obtaining a corresponding smoothed gradient sequence; if the spike is greater than a preset hyperparameter, finding a large gradient concentration interval; and selecting concentration test points by random uniform sampling according to weights; and selecting concentration test points from all other concentration intervals in the smoothed gradient sequence by random uniform sampling.

### CROSS REFERENCE OF RELATED APPLICATION

The present invention claims priority under 35 U.S.C. 119(a-d) to CN 202310183544.5, filed Mar. 1, 2023.

### BACKGROUND OF THE PRESENT INVENTION

### Field of Invention

The present invention relates to a technical field of gas sensor measurement, and more particularly to a calibration concentration selection method for a gas sensor array.

### Description of Related Arts

With the development of society, gas mixture detection is more and more widely used in manufacturing and daily life. During identification of gas mixtures, it is not only necessary to get the identification results of each gas, but also need to get the concentration of each gas in the gas mixture at one time. Gas mixture detection has important applications in the fields of chemical industry, electrical power, gas transmission, biochemical research, chemical pharmaceuticals, and detection of environmental pollution gases.

Conventional research on gas mixture detection algorithms mostly focuses on the artificial intelligence, and uses the priori data, which are obtained from testing, as features for model training and learning. Finally, the gas concentration of unknown samples is predicted through the model, which puts high requirements on the testing of gas sensor arrays in order to obtain a high accuracy. Test scheme is the basic training set of the algorithm model, whose quality is of guiding significance to the algorithm model training and prediction effects. Conventionally, the test schemes for gas sensor arrays mainly include: (1) randomly sampling in a certain range to formulate a gas concentration test scheme; (2) uniformly traversing with a certain gradient size in a certain range to formulate a gas concentration test scheme; and (3) formulating a gas concentration test scheme based on an orthogonal table method in a certain range.

However, the above test schemes have the following problems: test samples of the random sampling is not uniform, resulting in serious model training underfitting; the use of uniform traversal will cause the number of test points to grow exponentially, and if the gas concentration to be tested spans a large range, a large number of test sample points will be generated, leading to a great pressure on the test; and the use of orthogonal tables cannot be well matched with the algorithmic model, and it is difficult to train the distribution pattern of the data, so the model recognition effect obtained by using the distribution pattern as a training set is insufficient.

### SUMMARY OF THE PRESENT INVENTION

Aiming at the above technical problems in the prior art, an object of the present invention is to provide a calibration concentration selection method for a gas sensor array, which ensures a uniform distribution of sample points, facilitates subsequent prediction model learning, and helps to improve accuracy of concentration prediction for gas mixture.

Accordingly, the present invention provides:
a calibration concentration selection method for a gas sensor array, comprising steps of:
step 1: using the gas sensor array to obtain source domain data of a gas mixture formed by n gases, wherein the source domain data comprise a concentration variation sequence of each gas in the gas mixture, and a response variation sequence of each gas in the gas mixture; the response variation sequence corresponds to the concentration variation sequence one by one;
step 2: constructing a gas mixture prediction model (AE-BP model) with an auto encoder network (AEN) and a fully connected neural network; using the response variation sequence as an input and an output of the AEN, and training the AEN; extracting effective features of the response variation sequence from a bottleneck layer of a trained AEN; then using the effective features as an input, and using the concentration variation sequence of the source domain data as a training target, so as to train the fully connected neural network; wherein a trained AE-BP model is formed by the trained AEN and a trained fully connected neural network;
step 3: constructing a variational auto-encoder (VAE); identically distributing the response variation sequence of the source domain data through the VAE for generating an identically distributed response variation sequence for each gas in the gas mixture, wherein a total number of response values is N;
step 4: inputting the identically distributed response variation sequence into the trained AE-BP model to output a predicted concentration variation sequence of each gas in the gas mixture, wherein a total number of concentration values is N; and then normalizing the predicted concentration variation sequence according to a desired concentration test range of each gas in a target domain, so as to generate a target concentration variation sequence for each gas;
step 5: sorting a target concentration variation sequence of a kth gas according to a concentration value, and k=1, 2, . . . , n; correspondingly sorting target concentration variation sequences of remaining n−1 gases and corresponding n-dimensional identically distributed response variation sequences, thereby obtaining a sorted concentration variation sequence of the kth gas and a corresponding n-dimensional sorted identically distributed response variation sequence; calculating a response gradient sequence of the n-dimensional sorted identically distributed response variation sequence to the sorted concentration variation sequence of the kth gas, so as to obtain n response gradient sequences corresponding to the kth gas; calculating n response gradient sequences for the target concentration variation sequence of each gas, so as to obtain n×n response gradient sequences in total;
step 6: processing each of the response gradient sequences with absolute value calculation and sliding window filtering with a size of T, thereby obtaining a corresponding smoothed gradient sequence; wherein there are totally n×n smoothed gradient sequences;
step 7: calculating a spike of the smoothed gradient sequence, and if the spike is greater than a preset hyperparameter, executing step 8 to find a large gradient concentration interval of the corresponding smoothed gradient sequence; otherwise, executing step 9 for point selection;
step 8: dividing the smoothed gradient sequence, whose spike is greater than the hyperparameter, into P equal parts according to a gradient value, so as to obtain P−1 equal gradient values; traversing through the equal gradient values, and using a maximum entropy thresholding algorithm to calculate an upper region gradient value information entropy and a lower region gradient value information entropy with the gradient value as a dividing line; using an equal gradient value, with which a sum of the upper region gradient value information entropy and the lower region gradient value information entropy is maximized, as a threshold line; then taking a concentration interval in the smooth gradient sequence, which is larger than the threshold line, as a large gradient concentration interval, and obtaining no less than one large gradient concentration interval; then executing step 9 for point selection; and
step 9: assuming that a total number of test points required in the target domain is M, wherein there are M/2 test points in the large gradient concentration interval as well as in all other concentration intervals in the smoothed gradient sequence other than the large gradient concentration interval; weighting the large gradient concentration interval based on a corresponding maximum gradient value, then assigning the test points to the large gradient concentration interval according to weights, and selecting M/2 corresponding concentration test points by random uniform sampling; and selecting M/2 corresponding concentration test points from all other concentration intervals in the smoothed gradient sequence other than the large gradient concentration interval by random uniform sampling.

Preferably, n≤4.

Preferably, in step 4, a normalization formula is:

X′ₗ^{(k)}=a^{(k)}+[(b^{(k)}−a^{(k)})/(Max^{(k)}−Min^{(k)})]×(Xₗ^{(k)}−Min^{(k)})
wherein a^{(k)} and b^{(k)} are respectively minimum and maximum values of the desired concentration test range of the kth gas in the target domain, and k=1, 2, . . . , n; Min^{(k)} and Max^{(k)} are respectively minimum and maximum values of the predicted concentration variation sequence of the kth gas, and k=1, 2, . . . , n; Xₗ^{(k)} is an lth concentration value in the predicted concentration variation sequence of the kth gas, k=1, 2, . . . , n and l=1, 2, . . . , n; x′ₗ^{(k)} is an lth concentration value in the target concentration variation sequence of the kth gas, k=1, 2, . . . , n and l=1, 2, . . . , N.

Preferably, in step 5, the response gradient sequence is specifically a jth response gradient sequence G^{(kj)} corresponding to the kth gas, consisting of N−4 response gradient points Gᵢ^{(kj)}, wherein i=3, 4, . . . , N−2; k=1, 2, . . . , n; and j=1, 2, . . . , n; Gᵢ^{(kj)} is calculated by:

Gᵢ^{(kj)}=[−yᵢ₊₂^{(kj)}+8yᵢ₊₁^{(kj)}−8yᵢ₋₁^{(kj)}+yᵢ₋₂^{(kj)}]/[12×(xᵢ₊₁^{(k)}−xᵢ^{(k)})]
wherein xᵢ^{(k)} and xᵢ₊₁^{(k)} are respectively an ith concentration value and an (i+1)th concentration value of the kth gas in the sorted concentration variation sequence, and i=3, 4, . . . , N−2; yᵢ₊₂^{(kj)}, yᵢ₊₁^{(kj)}, yᵢ₋₁^{(kj)} and yᵢ₋₂^{(kj)} are respectively (i+2)th, (i+1)th, (i−1)th and (i−2)th response values in a jth-dimensional sorted identically distributed response variation sequence corresponding to the kth gas, i=3, 4, . . . , N−2; k=1, 2, . . . , n; and j=1, 2, . . . , n.

Preferably, in step 6, T is 50-100.

Preferably, in step 7, the hyperparameter is 10-15.

Preferably, in step 8, P is 10-30.

Preferably, in step 9, M is 50-1000.

The beneficial effects of the present invention are as follows.

1, The present invention proposes the calibration concentration selection method for the gas sensor array. The VAE-based method generates identically distributed data points according to the response variation sequence of the source domain data, which can better fill in the gaps in the source domain data, making the concentration test points more uniform without affecting the data distribution characteristics. Such scheme is superior to the gradient test scheme having larger data distribution vacancies, and at the same time avoids the excessive test pressure caused by the exponential growth of the test points with the gas types in the traversal test scheme.

2, The concentration test point obtained by the present invention are uniformly and intensively sampled from the high concentration interval with large response gradient, and uniformly and sparsely sampled from the low concentration interval with gentle response gradient, so as to obtain the concentration test scheme for the calibration of the concentration points. The calibration point strengthens the characteristics of data distribution in the source domain, which is used as the target domain to conduct gas test experiment. As a result, the distribution characteristics of the response value obtained by the gas sensor array are more obvious, which is more conducive to the gradient descent training of the gas mixture prediction model, thereby obtaining more accurate prediction results. Overall, since the VAE fills in the gaps in the data space as much as possible and intensively samples from the large gradient concentration interval, the trained gas mixture prediction model has strong generalization ability, and the prediction ability of the unknown samples is better.

### BRIEF DESCRIPTION OF THE DRAWINGS

FIG. 1 is a structural view of a gas mixture prediction model in a calibration concentration selection method for a gas sensor array according to an embodiment 1;

FIG. 2 illustrates relative error confidence intervals of the gas mixture prediction model in the calibration concentration selection method for gas sensor array according to the embodiment 1;

FIG. 3 illustrates 4 response gradient sequences of ammonia according to the embodiment 1;

FIG. 4 is a sketch view of maximum entropy threshold segmentation of a smoothed gradient sequence of nitrogen dioxide response among 4 smoothed gradient sequences of ammonia according to the embodiment 1;

FIG. 5 is a sketch view of maximum entropy threshold segmentation of a smoothed gradient sequence of ammonia response among 4 smoothed gradient sequences of ammonia according to the embodiment 1; and

FIG. 6 is a 3-dimensional feature point distribution of M test points obtained in the embodiment 1 after constant distance dimensionality reduction with a t-stochastic neighbor embedding (TSNE) dimensionality reduction algorithm.

### DETAILED DESCRIPTION OF THE PREFERRED EMBODIMENT

In order to make the objects, technical solutions and advantages of the present invention clearer and more understandable, the present invention will be further described below in conjunction with the accompanying drawings and embodiment. It should be understood that the preferred embodiment described herein is exemplary only and not intended to be limiting.

### Embodiment 1

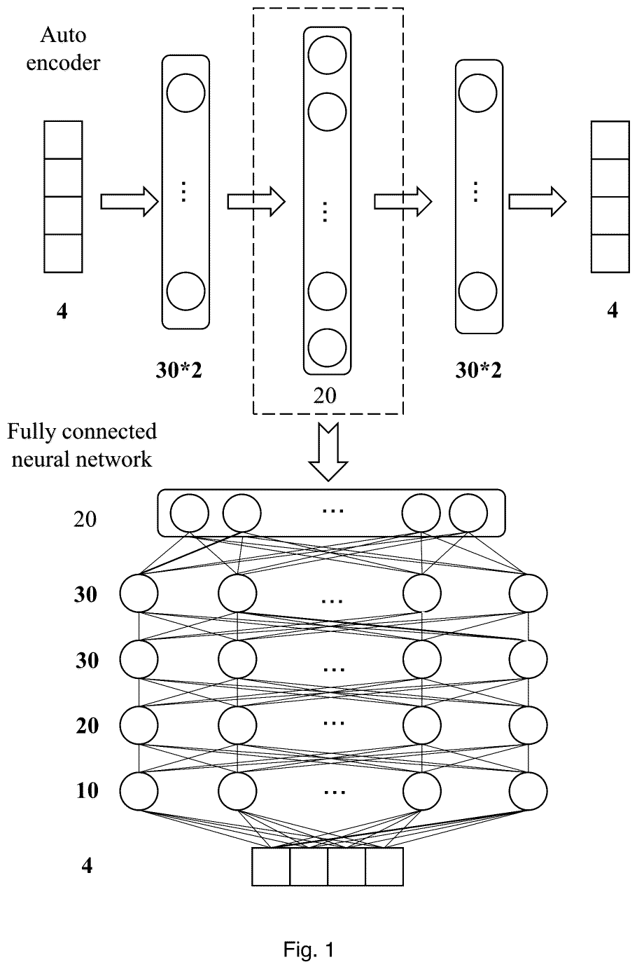

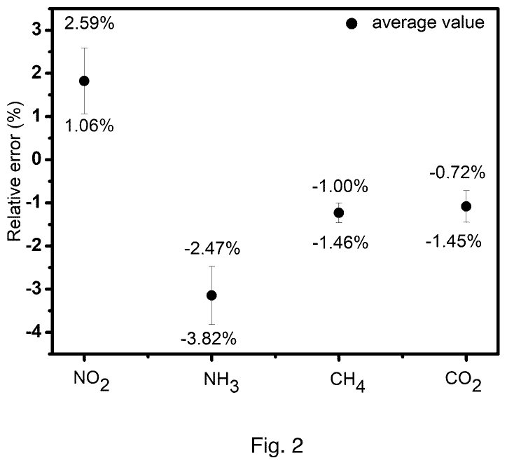

The embodiment 1 provides a calibration concentration selection method for a gas sensor array, comprising steps of:
step 1: using the gas sensor array to obtain source domain data of a gas mixture formed by 4 gases (nitrogen dioxide, ammonia, methane, and carbon dioxide), wherein the source domain data comprise a concentration variation sequence of each gas in the gas mixture, and a response variation sequence of each gas in the gas mixture; the response variation sequence corresponds to the concentration variation sequence one by one;
step 2: constructing an AE-BP model with an AEN and a fully connected neural network, as shown in FIG. 1;
wherein the AEN comprises:
1 input layer with 4 neurons;
5 hidden layers with 30, 30, 20, 30 and 30 neurons for each layer, respectively;
1 output layer with 4 neurons;
wherein activation functions of the input and hidden layers are both linear rectifier function (relu) functions, and no activation function is used in the output layer; the activation functions, optimizer, and network structure of the AEN are obtained by grid search and 3-fold cross-validation, and are based on a minimum loss in the case that the model is not overfitted;
wherein the fully connected neural network comprises:
1 input layer with 20 neurons;
4 hidden layers with 30, 30, 20 and 10 neurons for each layer, respectively;
1 output layer with 4 neurons;
wherein activation functions of the input and hidden layers are both relu functions, and no activation function is used in the output layer; the activation functions, optimizer, and network structure of the fully connected neural network are obtained by grid search and 3-fold cross-validation, and are based on a minimum loss in the case that the model is not overfitted;
using the response variation sequence as an input and an output of the AEN, and training the AEN; extracting effective features of the response variation sequence from a bottleneck layer of a trained AEN; then using the effective features as an input, and using the concentration variation sequence of the source domain data as a training target, so as to train the fully connected neural network; wherein a trained AE-BP model is formed by the trained AEN and a trained fully connected neural network;
wherein according to the embodiment 1, confidence intervals of the trained AE-BP model with relative errors of 95% confidence level are shown in FIG. 2, and it can be seen that none of the relative errors exceeds 4%, indicating model fitting accuracy is high;
step 3: constructing a VAE;
wherein the VAE comprises:
1 input layer with 4 neurons;
5 hidden layers with 20, 20, 15, 20 and 20 neurons for each layer, respectively;
1 output layer with 4 neurons;
wherein activation functions of the input and hidden layers are both relu functions, a bottleneck layer is normally distributed sample points, and no activation function is used in the output layer; the activation functions, optimizer, and network structure of the VAE are obtained by grid search and 3-fold cross-validation, and are based on a minimum loss function value;
identically distributing the response variation sequence of the source domain data through the VAE for generating an identically distributed response variation sequence for each gas in the gas mixture, wherein a total number of response values is N=3500;
step 4: inputting the identically distributed response variation sequence into the trained AE-BP model to output a predicted concentration variation sequence of each gas in the gas mixture, wherein a total number of concentration values is N=3500; and then normalizing the predicted concentration variation sequence according to a desired concentration test range of each gas in a target domain, so as to generate a target concentration variation sequence for each gas;
wherein a normalization formula is:

X′ₗ^{(k)}=a^{(k)}+[(b^{(k)}−a^{(k)})/(Max^{(k)}−Min^{(k)})]×(Xᵢ^{(k)}−Min^{(k)})
wherein a^{(k)} and b^{(k)} are respectively minimum and maximum values of the desired concentration test range of the kth gas in the target domain, and k=1, 2, 3, 4; Min^{(k)} and Max^{(k)} are respectively minimum and maximum values of the predicted concentration variation sequence of the kth gas, and k=1, 2, 3, 4; Xₗ^{(k)} is an lth concentration value in the predicted concentration variation sequence of the kth gas, k=1, 2, 3, 4 and l=1, 2, . . . , N; X′ₗ^{(k)} is an lth concentration value in the target concentration variation sequence of the kth gas, k=1, 2, 3, 4 and l=1, 2, . . . , N;
step 5: sorting a target concentration variation sequence of a kth gas according to a concentration value, and k=1, 2, 3, 4; correspondingly sorting target concentration variation sequences of remaining 3 gases and corresponding 4-dimensional identically distributed response variation sequences, thereby obtaining a sorted concentration variation sequence of the kth gas and a corresponding 4-dimensional sorted identically distributed response variation sequence; calculating a response gradient sequence of the 4-dimensional sorted identically distributed response variation sequence to the sorted concentration variation sequence of the kth gas, so as to obtain 4 response gradient sequences corresponding to the kth gas; calculating 4 response gradient sequences for the target concentration variation sequence of each gas, so as to obtain 4×4=16 response gradient sequences in total;
wherein the response gradient sequence is specifically a jth response gradient sequence G^{(kj)} corresponding to the kth gas, consisting of N−4 response gradient points Gᵢ^{(k)}, wherein i=3, 4, . . . , N−2; k=1, 2, 3, 4; and j=1, 2, 3, 4; Gᵢ^{(kj)} is calculated by:

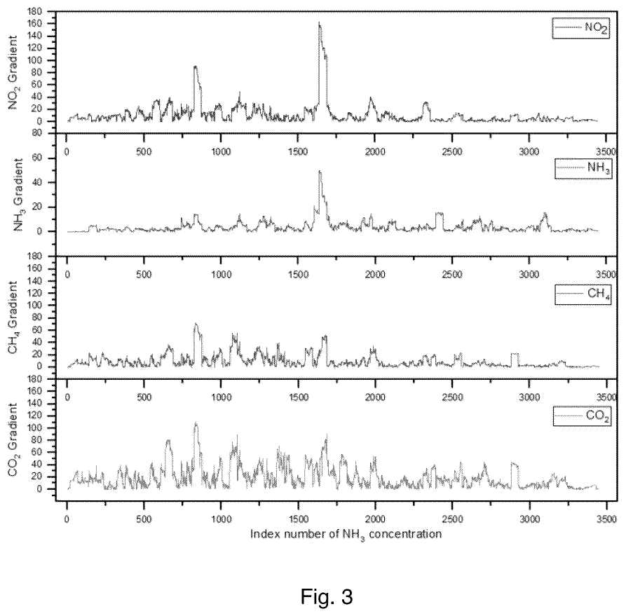

Gᵢ^{(k)}=[−yᵢ₊₂^{(kj)}+8yᵢ₊₁^{(kj)}−8yᵢ₋₁^{(kj)}+yᵢ₋₂^{(kj)}]/[12×(xᵢ₊₁^{(kj)}−xᵢ^{(k)})]
wherein xᵢ^{(k)} and xᵢ₊₁^{(k)} are respectively an ith concentration value and an (i+1)th concentration value of the kth gas in the sorted concentration variation sequence, and i=3, 4, . . . , N−2; yᵢ₊₂^{(kj)}, yᵢ₊₁^{(kj)}, yᵢ₋₁^{(kj)} and yᵢ₋₂^{(kj)} are respectively (i+2)th, (i+1)th, (i−1)th and (i−2)th response values in a jth-dimensional sorted identically distributed response variation sequence corresponding to the kth gas, i=3, 4, . . . , N−2; k=1, 2, 3, 4; and j=1, 2, 3, 4;
wherein in the embodiment 1, 4 response gradient sequences corresponding to ammonia are shown in FIG. 3, i.e., the response gradient sequences of the sorted identically distributed response variation sequences of nitrogen dioxide, ammonia, methane, and carbon dioxide to the sorted concentration variation sequences of ammonia;
step 6: processing each of the response gradient sequences with absolute value calculation and sliding window filtering with T=50, thereby obtaining a corresponding smoothed gradient sequence; i.e., an jth smoothed gradient sequence G′^{(kj)} corresponding to the kth gas, k=1, 2, 3, 4 and j=1, 2, 3, 4; there are totally 16 smoothed gradient sequences;
step 7: calculating a spike SP of the smoothed gradient sequence:

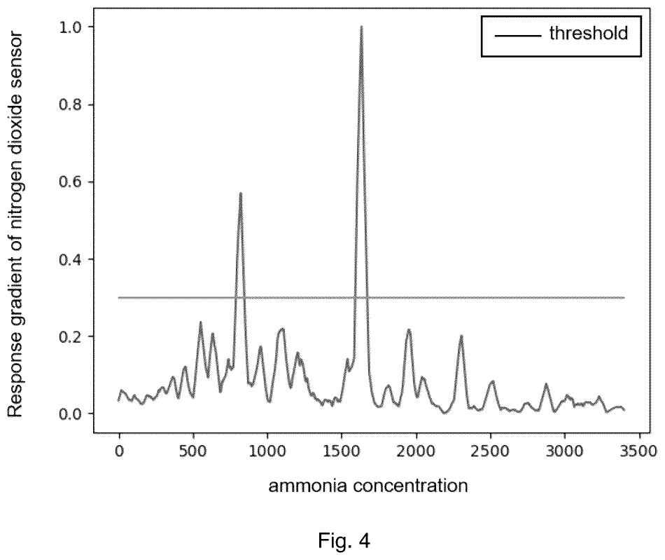

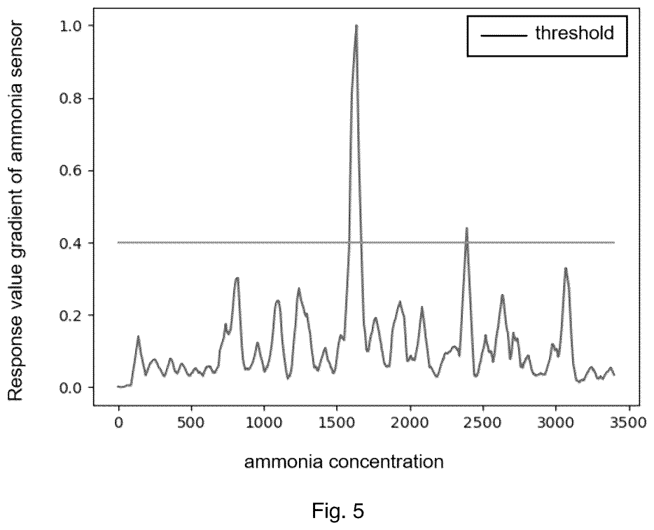

SP=[(N−4)×Max(G′^{(kj)})]/Σ(G′ᵢ^{(kj)})
wherein Max(G′^{(kj)}) is a maximum gradient value in G′^{(kj)}, k=1, 2, 3, 4 and j=1, 2, 3, 4; Σ(G′ᵢ^{(kj)}) is a sum of all gradient values in G′^{(kj)}, i=3, 4, . . . , N−2; k=1, 2, 3, 4; and j=1, 2, 3, 4;
wherein in the embodiment 1, the spikes SP of the four smoothed gradient sequences corresponding to ammonia are 14.039 (nitrogen dioxide), 12.311 (ammonia), 7.304 (methane), and 5.686 (carbon dioxide), respectively;
if the spike SP is greater than a preset hyperparameter of 10, executing step 8 to find a large gradient concentration interval of the corresponding smoothed gradient sequence; otherwise, executing step 9 for point selection;
step 8: dividing the smoothed gradient sequence, whose spike is greater than the hyperparameter, into P=10 equal parts according to a gradient value, so as to obtain P−1=9 equal gradient values; traversing through the equal gradient values, and using a maximum entropy thresholding algorithm to calculate an upper region gradient value information entropy and a lower region gradient value information entropy with the gradient value as a dividing line; using an equal gradient value, with which a sum of the upper region gradient value information entropy and the lower region gradient value information entropy is maximized, as a threshold line; then taking a concentration interval in the smooth gradient sequence, which is larger than the threshold line, as a large gradient concentration interval, and obtaining no less than one large gradient concentration interval; then executing step 9 for point selection;
wherein in the embodiment 1, among the 4 smoothed gradient sequences corresponding to ammonia, the smoothed gradient sequence of nitrogen dioxide response and the smoothed gradient sequence of ammonia response have a spike SP greater than the hyperparameter 10; the smoothed gradient sequences are processed with min-max normalization, and the threshold lines calculated according to the maximum entropy thresholding algorithm are shown in FIGS. 4 and 5; and
step 9: assuming that a total number of test points required in the target domain is M=120, wherein there are M/2=60 test points in the large gradient concentration interval as well as in all other concentration intervals in the smoothed gradient sequence other than the large gradient concentration interval; weighting the large gradient concentration interval based on a corresponding maximum gradient value, then assigning the test points to the large gradient concentration interval according to weights, and selecting M/2=60 corresponding concentration test points by random uniform sampling; and selecting M/2=60 corresponding concentration test points from all other concentration intervals in the smoothed gradient sequence other than the large gradient concentration interval by random uniform sampling.

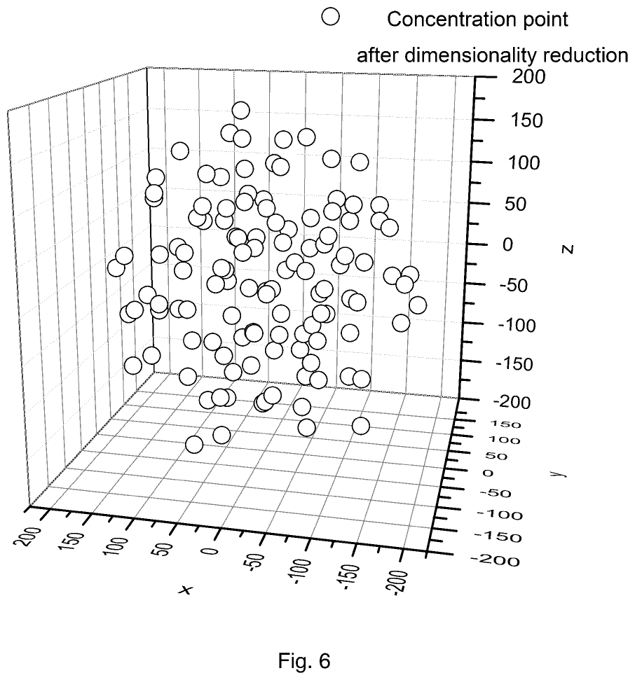

FIG. 6 a 3-dimensional feature point distribution of 120 concentration calibration points obtained in the embodiment 1 after constant distance dimensionality reduction with a TSNE dimensionality reduction algorithm. It can be seen that the concentration calibration points are uniformly distributed in the 3-dimensional space, which indicates that the calibration concentration selection method in the embodiment 1 can calibrate the test concentration points as uniformly as possible with a small number of calibrations, so as to fill in the gaps in the gradient test scheme caused by data distribution, which is conducive to the subsequent prediction model training.

The foregoing is only one preferred embodiment of the present invention. Any of the features disclosed in this specification, unless specifically recited, may be replaced by other equivalent or alternative features having a similar purpose. All of the features disclosed, or all of the steps in the method or process, may be combined in any manner, except for features and/or steps that are mutually exclusive.

What is claimed is:

1. A calibration concentration selection method for a gas sensor array, comprising steps of:
step 1: using the gas sensor array to obtain source domain data of a gas mixture formed by n gases, wherein the source domain data comprise a concentration variation sequence of each gas in the gas mixture, and a response variation sequence of each gas in the gas mixture;
step 2: constructing a gas mixture prediction model (AE-BP model) with an auto encoder network (AEN) and a fully connected neural network; using the response variation sequence as an input and an output of the AEN, and training the AEN; extracting effective features of the response variation sequence from a bottleneck layer of a trained AEN; then using the effective features as an input, and using the concentration variation sequence of the source domain data as a training target, so as to train the fully connected neural network; wherein a trained AE-BP model is formed by the trained AEN and a trained fully connected neural network;
step 3: constructing a variational auto-encoder (VAE); identically distributing the response variation sequence of the source domain data through the VAE for generating an identically distributed response variation sequence for each gas in the gas mixture, wherein a total number of response values is N;
step 4: inputting the identically distributed response variation sequence into the trained AE-BP model to output a predicted concentration variation sequence of each gas in the gas mixture, wherein a total number of concentration values is N; and then normalizing the predicted concentration variation sequence according to a desired concentration test range of each gas in a target domain, so as to generate a target concentration variation sequence for each gas;
step 5: sorting a target concentration variation sequence of a kth gas according to a concentration value, and k=1, 2, . . . , n; correspondingly sorting target concentration variation sequences of remaining n−1 gases and corresponding n-dimensional identically distributed response variation sequences, thereby obtaining a sorted concentration variation sequence of the kth gas and a corresponding n-dimensional sorted identically distributed response variation sequence; calculating a response gradient sequence of the n-dimensional sorted identically distributed response variation sequence to the sorted concentration variation sequence of the kth gas, so as to obtain n response gradient sequences corresponding to the kth gas; calculating n response gradient sequences for the target concentration variation sequence of each gas, so as to obtain n×n response gradient sequences in total;
step 6: processing each of the response gradient sequences with absolute value calculation and sliding window filtering with a size of T, thereby obtaining a corresponding smoothed gradient sequence;
step 7: calculating a spike of the smoothed gradient sequence, and if the spike is greater than a preset hyperparameter, executing step 8 to find a large gradient concentration interval of the corresponding smoothed gradient sequence; otherwise, executing step 9 for point selection;
step 8: dividing the smoothed gradient sequence, whose spike is greater than the hyperparameter, into P equal parts according to a gradient value, so as to obtain P−1 equal gradient values; traversing through the equal gradient values, and using a maximum entropy thresholding algorithm to calculate an upper region gradient value information entropy and a lower region gradient value information entropy with the gradient value as a dividing line; using an equal gradient value, with which a sum of the upper region gradient value information entropy and the lower region gradient value information entropy is maximized, as a threshold line; then taking a concentration interval in the smooth gradient sequence, which is larger than the threshold line, as a large gradient concentration interval, and obtaining no less than one large gradient concentration interval; then executing step 9 for point selection; and
step 9: assuming that a total number of test points required in the target domain is M, wherein there are M/2 test points in the large gradient concentration interval as well as in all other concentration intervals in the smoothed gradient sequence other than the large gradient concentration interval; weighting the large gradient concentration interval based on a corresponding maximum gradient value, then assigning the test points to the large gradient concentration interval according to weights, and selecting M/2 corresponding concentration test points by random uniform sampling; and selecting M/2 corresponding concentration test points from all other concentration intervals in the smoothed gradient sequence other than the large gradient concentration interval by random uniform sampling.

2. The calibration concentration selection method, as recited in claim 1, wherein in step 4, a normalization formula is:
X′ₗ^{(k)}=a^{(k)}+[(b^{(k)}−a^{(k)})/(Max^{(k)}−Min^{(k)})]×(Xₗ^{(k)}−Min^{(k)})
wherein a^{(k)} and b^{(k)} are respectively minimum and maximum values of the desired concentration test range of the kth gas in the target domain, and k=1, 2, . . . , n; Min^{(k)} and Max^{(k)} are respectively minimum and maximum values of the predicted concentration variation sequence of the kth gas, and k=1, 2, . . . , n; Xₗ^{(k)} is an lth concentration value in the predicted concentration variation sequence of the kth gas, k=1, 2, . . . , n and l=1, 2, . . . , N; X′ₗ^{(k)} is an lth concentration value in the target concentration variation sequence of the kth gas, k=1, 2, . . . , n and l=1, 2, . . . , N.

3. The calibration concentration selection method, as recited in claim 1, wherein in step 5, the response gradient sequence is specifically a jth response gradient sequence G^{(kj)} corresponding to the kth gas, consisting of N−4 response gradient points Gᵢ^{(kj)}, wherein i=3, 4, . . . , N−2; k=1, 2, . . . , n; and j=1, 2, . . . , n; Gᵢ^{(kj)} is calculated by:
Gᵢ^{(k)}=[−yᵢ₊₂^{(kj)}+8yᵢ₊₁^{(kj)}−8yᵢ₋₁^{(kj)}+yᵢ₋₂^{(kj)}]/[12×(xᵢ₊₁^{(k)}−xᵢ^{(k)})]
wherein xᵢ^{(k)} and xᵢ₊₁^{(k)} are respectively an ith concentration value and an (i+1)th concentration value of the kth gas in the sorted concentration variation sequence, and i=3, 4, . . . , N−2; yᵢ₊₂^{(kj)}, yᵢ₊₁^{(kj)}, yᵢ₋₁^{(kj)} and yᵢ₋₂^{(kj)} are respectively (i+2)th, (i+1)th, (i−1)th and (i−2)th response values in a jth-dimensional sorted identically distributed response variation sequence corresponding to the kth gas, i=3, 4, . . . , N−2; k=1, 2, . . . , n; and j=1, 2, . . . , n.

4. The calibration concentration selection method, as recited in claim 1, wherein n≤4.

5. The calibration concentration selection method, as recited in claim 1, wherein in step 6, T is 50-100.

6. The calibration concentration selection method, as recited in claim 1, wherein in step 7, the hyperparameter is 10-15.

7. The calibration concentration selection method, as recited in claim 1, wherein in step 8, P is 10-30.

8. The calibration concentration selection method, as recited in claim 1, wherein in step 9, M is 50-1000.
